(19) United States
(12) Patent Application Publication
(10) Pub. No.: US 2010/0161058 A1
(43) Pub. Date: Jun. 24, 2010

(54) Multiple-State Geometry Artificial Disc With Compliant Insert and Method

(75) Inventors: Mahmoud F. Abdelgany, Rockaway, NJ (US); Aaron D. Markworth, Saddle Brook, NJ (US); Young Hoon Oh, Montville, NJ (US)

Correspondence Address: Rahman LLC, 10025 Governor Warfield Parkway, Suite 110, Columbia, MD, 21044, US

(73) Assignee: Custom Spine, Inc., Parsippany, NJ (US)

(21) Appl. No.: 12/344,212

(22) Filed: Dec. 24, 2008

(51) Int. Cl. A61F 2/44

(52) U.S. Cl. 623/17.16

(57) ABSTRACT

A multiple-state geometry artificial disc assembly attached to vertebrae includes a compliant load bearing spacer element having an upper curved portion and a lower curved portion, a first plate coupled on the upper curved portion of the compliant load bearing spacer element and a second plate coupled on the lower curved portion of the compliant load bearing spacer element. The first plate and the second plate preferably are of a flexible material. The first plate and the second plate transitions from a convex configuration to a concave configuration in-situ in a vertebral disc space. The upper curved portion and the lower curved portion of the compliant load bearing spacer element may include a plurality of openings. The compliant load bearing spacer element may further include a middle cylindrical portion dimensioned and configured to match a gap between the first plate and the second plate.

### BACKGROUND

1. Technical Field

The embodiments herein generally relate to medical disc implants, and more particularly, to a multiple-state geometry artificial disc with compliant insert used during disc replacement surgeries.

2. Description of the Related Art

Intervertebral discs lie between adjacent vertebrae in the human spine. Each disc forms a cartilaginous joint to allow slight movement of the vertebrae, and acts as a ligament to hold the vertebrae together. The intervertebral discs contain an outer annulus fibrosus, which surrounds the inner nucleus pulposus. The nucleus pulposus acts as a shock absorber, absorbing the impact of the body's daily activities and keeping the two vertebrae separated. When one develops a prolapsed disc the nucleus pulposus is forced out of the disc and may put pressure on the nerve located near the disc. Gradual dehydration of the nucleus pulposus leads to degenerative disc disease.

When the annulus fibrosus tears due to an injury or the aging process, the nucleus pulposus can begin to extrude through the tear. This is called disc herniation. Artificial disc replacement is a surgical procedure in which degenerated intervertebral discs are replaced with artificial ones. The procedure is primarily used to treat chronic, severe low back pain and cervical pain resulting from degenerative disc disease. In one technique of artificial disc replacement, flexible discs are placed within the intervertebral disc space without any anchoring system with the expectation that it will remain in place based on contact with the ligaments of the disc annulus or the vertebral bodies. This approach tends to have either a spring or damping effect or control of rotation but not both at the same time. Another approach is to have two vertebral bodies bound with some elastic nucleus. This approach has translation with a spring/damping effect. Alternative approaches include shell shaped devices with a spacer in-between. The upper and lower shells include a pair of interconnected cylindrical lobes. These devices are difficult to implant and revise due to endplate damage. In addition, they suffer from a lack of natural movement and have uncontrolled movement.

### SUMMARY

In view of the foregoing, an embodiment herein provides a multiple-state geometry artificial disc assembly attached to vertebrae. The multiple-state geometry artificial disc assembly includes a compliant load bearing spacer element having an upper curved portion and a lower curved portion, a first plate coupled on the upper curved portion of the compliant load bearing spacer element and a second plate coupled on the lower curved portion of the compliant load bearing spacer element. The first plate and the second plate include a flexible material. The first plate and the second plate transitions from a convex configuration to a concave configuration in-situ in a vertebral disc space.

The upper curved portion and the lower curved portion of the compliant load bearing spacer element may include a plurality of openings. The compliant load bearing spacer element may further include a middle cylindrical portion dimensioned and configured to match a gap between the first plate and the second plate. The first plate and the second plate include a plurality of spikes on at least one surface of the first plate and the second plate. The spikes embed into the vertebrae. The flexible material preferably is any of a polymer, a metal, and a nitinol shaped memory alloy. The compliant load bearing spacer element preferably is any of a flexible polymer material, a polymer, and a hydro-gel.

Another embodiment provides an apparatus to restore a spinal segment mobility. The apparatus includes a first plate having a flexible material, a second plate including flexible material, and a compliant load bearing spacer element positioned between the first plate and the second plate. The compliant load bearing spacer element includes an upper curved portion, a middle cylindrical portion, and a lower curved portion. Each of the first plate and the second plate includes a top surface including at least one spike extending outwardly from the top surface, a bottom surface, a wall configured around a circumference of the first plate and the second plate such that the wall separates the top surface from the bottom surface, and at least one gap dispersed along the wall.

The first plate and the second plate transition from a convex configuration to a concave configuration. The compliant load bearing spacer element may cause the transition of the first plate and the second plate from a convex configuration to a concave configuration to occur in-situ in a vertebral disc space. The compliant load bearing spacer element may control at least one of a rigid rotation, a translation, and an active spring plus damping of vertebral bodies. The compliant load bearing spacer element preferably includes a monolithic mass insert-molded around another body of a varied geometry.

The compliant load bearing spacer element includes a dual durometer material. The dual durometer material may control at least one of a flexion, an extension, a rotation, and a translation of vertebral bodies. The compliant load-bearing spacer element includes a plurality of openings. The first plate and the second plate are preferably any of a polymer, a metal, and a nitinol shaped memory alloy.

Yet embodiment provides a method of implanting an artificial vertebral disc. The method includes inserting a first plate having outwardly protruding spikes in a vertebral space and adjacent to a first endplate of a first vertebral body, inserting a second plate having outwardly protruding spikes in the vertebral space and adjacent to a second endplate of a second vertebral body such that a gap exists between the first plate and the second plate, and inserting a compliant load bearing spacer element in between the fist plate and the second plate in the vertebral space causing the first plate and the second plate to each transition into a concave configuration. The first plate are in a convex configuration.

The compliant load bearing spacer element includes an upper curved portion having a plurality of openings, a middle cylindrical portion dimensioned and configured to match a configuration of the gap between the first plate and second plate. A lower curved portion includes a plurality of openings. The first plate and second plate are preferably any of a polymer, a metal, and a nitinol shaped memory alloy.

The outwardly protruding spikes may be embedded into the first vertebral body and the second vertebral body when the first plate and the second plate are in the concave configuration. The compliant load bearing spacer element may control at least one of a rigid rotation, a translation, and an active spring plus damping of the first vertebral body and the second vertebral body. The compliant load bearing spacer element is preferably any of a flexible polymer material, a polymer, and a hydro-gel.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1A illustrates a front view of a pair of plates of a multiple-state geometry artificial disc implant during a first stage of insertion according to an embodiment herein;

FIG. 1B illustrates a front view of the pair of plates of FIG. 1A of a multiple-state geometry artificial disc implant during a second stage of insertion according to an embodiment herein;

FIG. 1C illustrates a front view of a third stage of insertion of a multiple-state geometry artificial disc implant according to an embodiment herein;

FIG. 2A illustrates a rotated front view of an assembled multiple-state geometry artificial disc implant of FIG. 1C configured in its third stage of insertion according to an embodiment herein;

FIG. 2B illustrates a rotated cross-sectional view of the assembled multiple-state geometry artificial disc implant of FIG. 2A configured in its third stage of insertion according to an embodiment herein;

FIG. 3A illustrates a perspective view of the top side of one of the plates of FIGS. 1A and 1B in a convex configuration according to an embodiment herein;

FIG. 3B illustrates a perspective view of the bottom side of the plate of FIG. 3A in a convex configuration according to an embodiment herein;

FIG. 3C illustrates a perspective view of the top side of one of the plates of FIG. 1C in a concave configuration according to an embodiment herein;

FIG. 3D illustrates a perspective view of the bottom side of the plate of FIG. 3C in a concave configuration according to an embodiment herein;

FIG. 4 illustrates a perspective view of the compliant load bearing spacer element of FIGS. 1C through 2B according to an embodiment herein; and

FIG. 5 is a flow diagram illustrating a preferred method of implanting an artificial vertebral disc according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

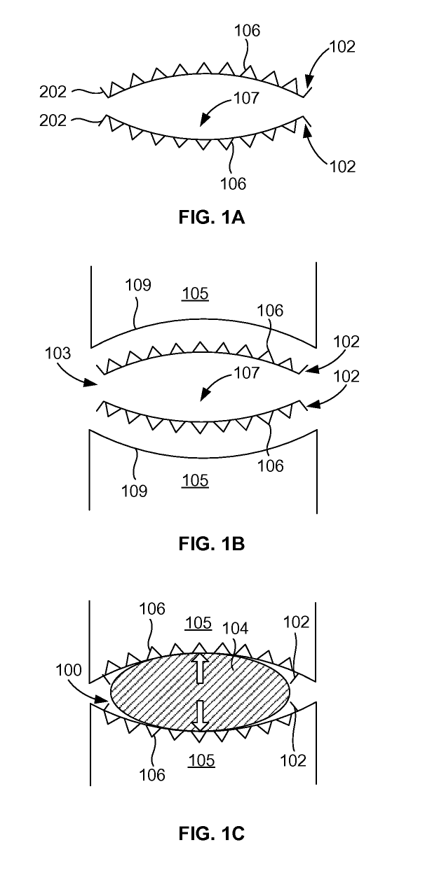

The embodiments herein provide a disc that can transition back and forth for ease of insertion followed by expansion in the vertebral disc space. This reduces the chance of damages to the soft tissue and the endplate of the vertebral bodies during implantation. Referring now to the drawings, and more particularly to FIGS. 1A through 5, where similar reference characters denote corresponding features consistently throughout the figures, there are shown preferred embodiments.

With respect to FIGS. 1A through 1C, FIG. 1A illustrates a front view of a pair of plates 102 of a multiple-state geometry artificial disc implant 100 during a first stage of insertion according to an embodiment herein, FIG. 1B illustrates a front view of the pair of plates 102 of FIG. 1A of a multiple-state geometry artificial disc implant 100 during a second stage of insertion according to an embodiment herein, and FIG. 1C illustrates a front view of a third stage of insertion of a multiple-state geometry artificial disc implant 100 according to an embodiment herein.

In a preferred embodiment, the plates 102 comprise a Nitinol shape memory alloy to allow it to deform and transition from a convex to a concave configuration. The plates 102 comprise a plurality of spikes 106 extending outwardly from the surface of one side of each plate 102. In FIGS. 1A through 1C, of the two plates 102, the top plate may be referred to as the superior plate while the bottom plate may be referred to as the inferior plate. However, the construction and configuration of both plates 102 are identical. During the initial stage of insertion, only the plates 102 are inserted into the disc space 103 in a convex shape. In this regard, the sharp spikes 106 are hidden in that they are not in contact with the vertebral bodies 105. In this way, one can avoid/minimize damage to the endplate surfaces 109 of the vertebrae 105. At the same time, the minimum opening space 103 allows for proper insertion of the plates 102. In one embodiment, the spikes 106 are embodied like cheese-grater like serrations on the plates 102. In alternative embodiments the serrations are shaped in the form of flanges. The compliant load bearing spacer element 104 constitutes a shape that is matched to the gap 107 between the plates 102.

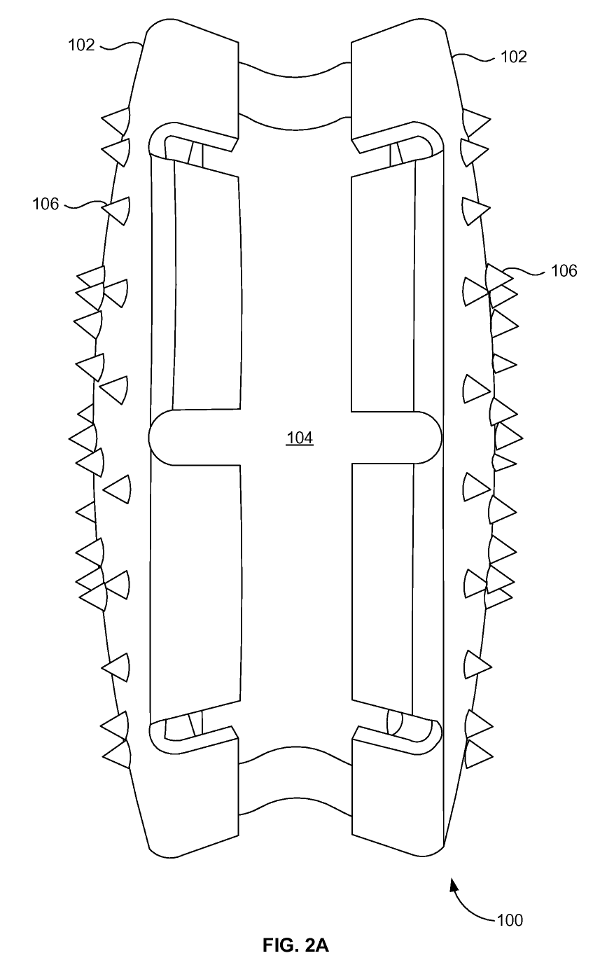

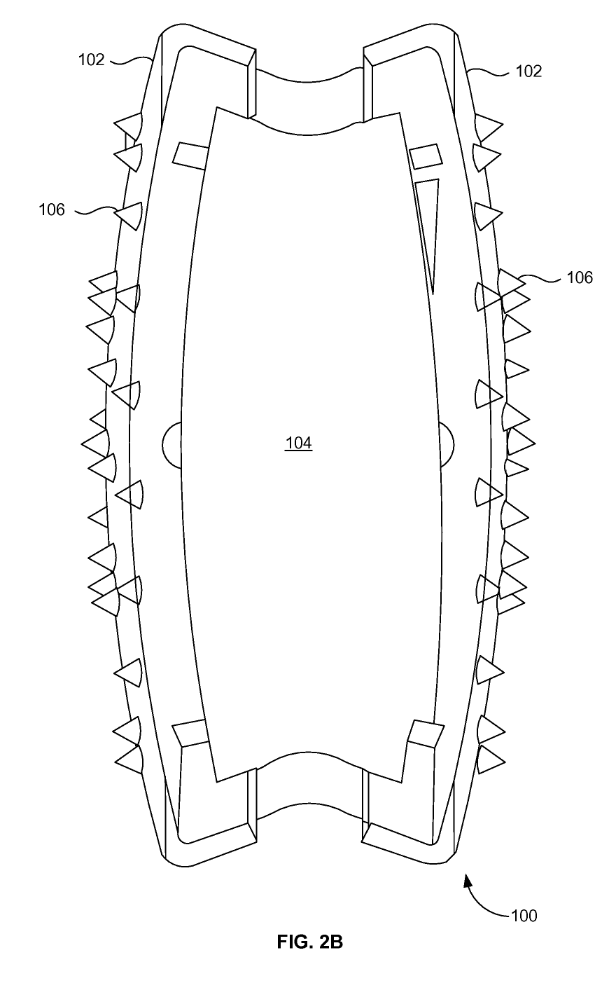

During the third stage of insertion, the plates 102 transform from a convex configuration to a concave configuration by insertion of a compliant load bearing spacer element 104 in between the plates 102. The spacer element 104 pushes against each plate 102 (as indicated by the block arrows in FIG. 1C) causing the deformable plates 102 to transform from convex to concave in shape, and thus, the spikes 106 engage and become embedded in the vertebrae 105. The final configuration of the implant 100 is illustrated in FIGS. 1C through 2B where FIG. 2A illustrates a rotated front view of an assembled multiple-state geometry artificial disc implant 100 of FIG. 1C configured in its third stage of insertion according to an embodiment herein, and FIG. 2B illustrates a rotated cross-sectional view of the assembled multiple-state geometry artificial disc implant of FIG. 2A configured in its third stage of insertion according to an embodiment herein;

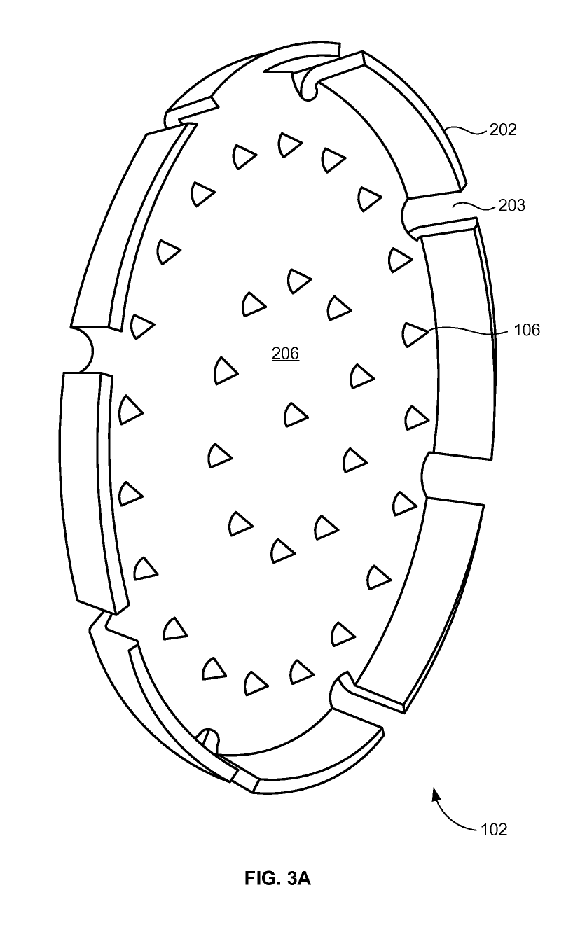

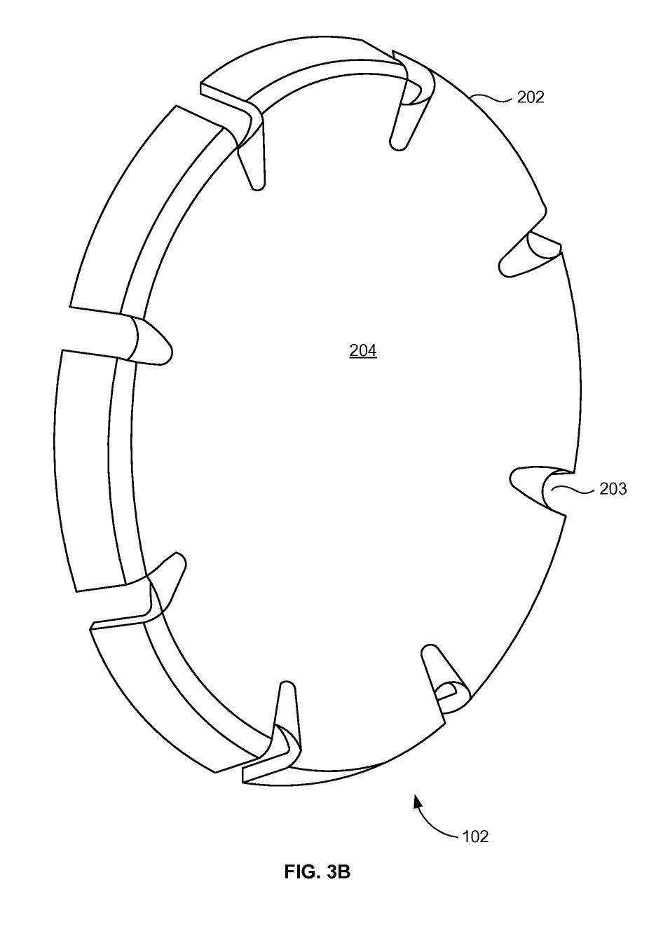

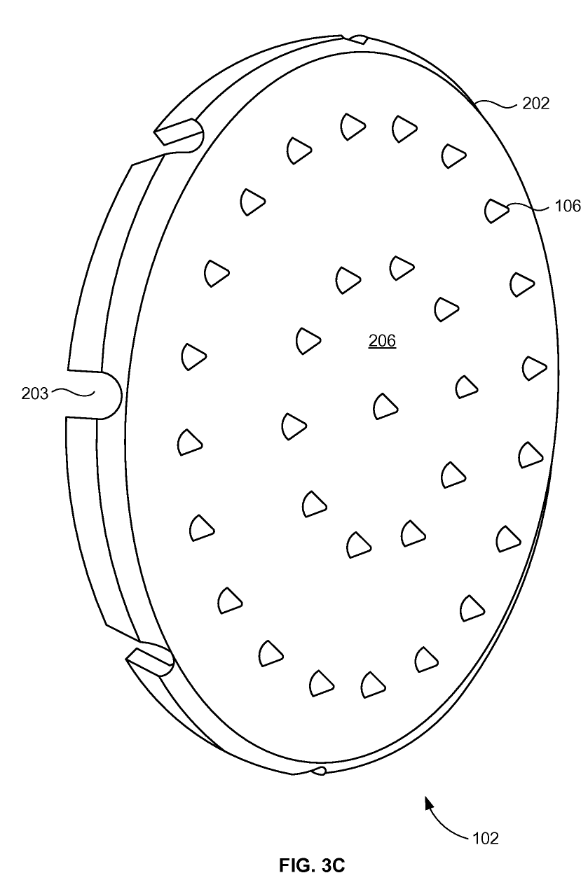

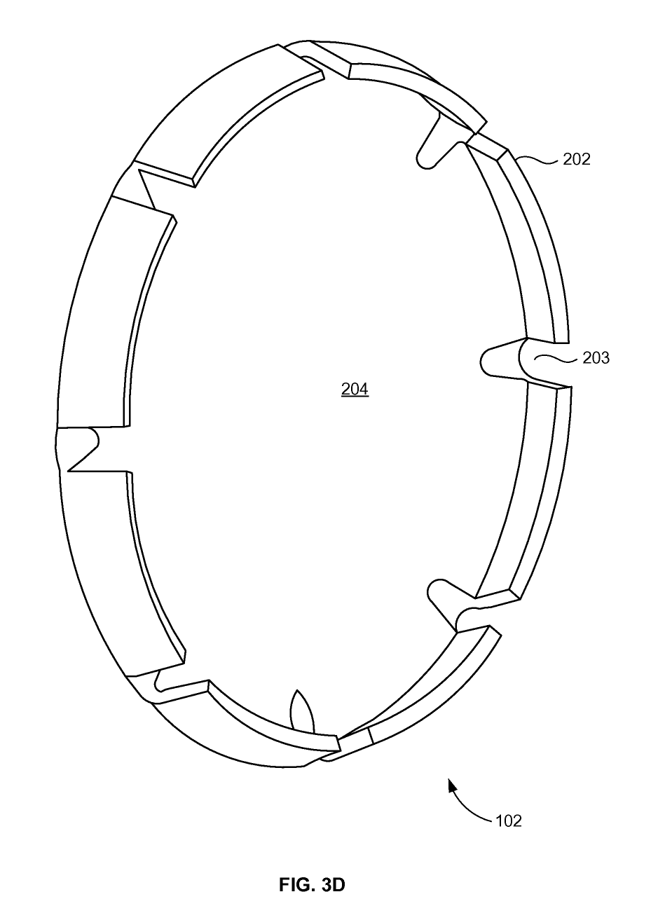

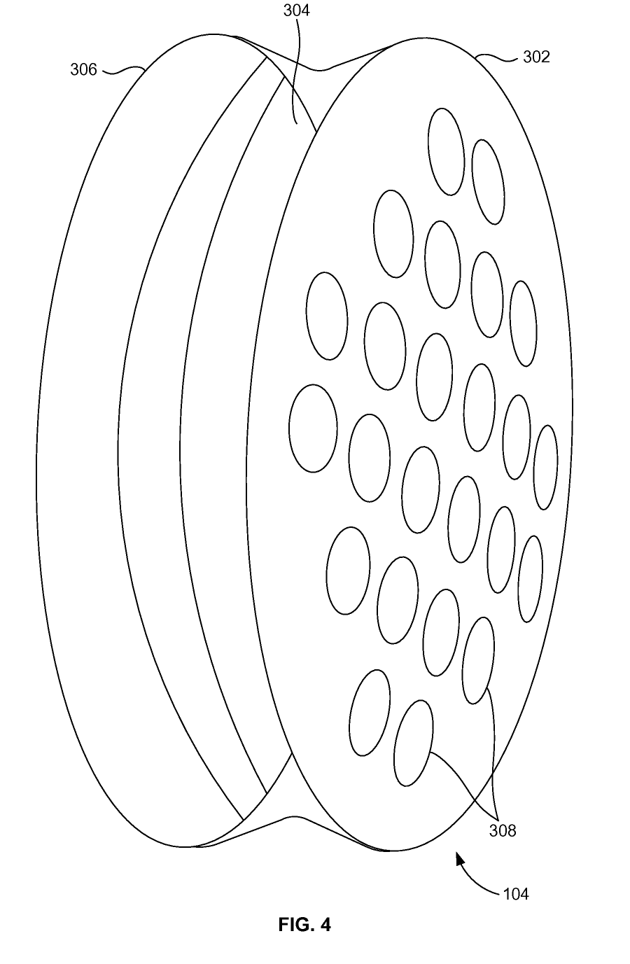

FIG. 3A illustrates a perspective view of the top side of one of the plates 102 of FIGS. 1A and 1B in a convex configuration according to an embodiment herein. The plate 102 is initially convex as indicated in FIGS. 1A and 1B and is transitioned to concave in-situ as indicated in FIG. 1C to match the endplate surfaces 109 of the vertebrae 105. In FIG. 3A, the spikes 106 are shown extending from the outer surface 206 of the convex plate 102. A circumferential wall 202 that is broken up by a series of gaps 203 extends around the outer circumference of the plate 102. FIG. 3B illustrates a perspective view of the bottom side of the plate 102 of FIG. 3A in a convex configuration according to an embodiment herein. The outer surface 204 of this side of the plate 102 does not contain any spikes 106.

FIG. 3C illustrates a perspective view of the top side of one of the plates 102 of FIG. 1C in a concave configuration according to an embodiment herein. The difference between FIGS. 3A and 3C is that FIG. 3A shows the plate 102 in a convex configuration, while FIG. 3C shows the plate 102 in a concave configuration. Moreover, FIG. 3D illustrates a perspective view of the bottom side of the plate 102 of FIG. 3C in a concave configuration according to an embodiment herein. Similarly, the difference between FIG. 3B and 3D is that FIG. 3B shows the plate 102 in a convex configuration, while FIG. 3D shows the plate 102 in a concave configuration.

FIG. 4 illustrates a perspective view of the compliant load bearing spacer element 104 of FIGS. 1C through 2B according to an embodiment herein. The spacer element 104 is positioned between the plates 102 during the third stage of insertion (FIG. 1C) to allow the plates 102 to transform from a convex configuration (FIGS. 1A, 1B, 3A, and 3B) to a concave configuration (FIG. 1C through 2B and FIGS. 3A through 3B). The spacer element 104 is inserted between the plates 102 to maintain vertebral disc height. The spacer element 104 is configured as a cylindrical disc-like structure having an upper curved portion 302, a middle cylindrical portion 304, and a lower curved portion 306. The upper curved portion 302 and lower curved portion 306 further includes a plurality of openings 308, which are configured to allow the spacer element 104, which is deformable, to be easily compressed and spring back to its original shape.

The middle cylindrical portion 304 of the spacer element 104 is dimensioned and configured to match the gap 107 between the plates 102 and to cushion the effect of the translation of the vertebral bodies 105 by absorbing contraction and expansion forces during the movement of the spine. Additionally, the spacer element 104 may comprise flexible polymer material, polymer, or hydro-gel, for example. The spacer element 104 also acts like the anatomical disc while controlling rigid rotation, translation, and active spring plus damping. If the plates 102 become too fixed too bone, a harder spacer (not shown) could be put in place to simulate fusion or rigid fixation providing an easier revision method versus current devices. In a preferred embodiment, the spacer element 104 can be a monolithic mass “insert-molded” around another body of varied geometry, as a means of controlling range of motion and compression of an assembled device.

In another preferred embodiment, instead of being “insert-molded” around another body, the spacer element 104 can be of a dual durometer material in the ventral-dorsal direction to shift the center of rotation to a more anatomically correct position. The material could have a plurality of transitions between the different durometer areas as deemed necessary to control flexion, extension, rotation, and translation anatomically. In another embodiment, the plates 102 may be connected with a plurality of pre-connected living hinges or connectors (not shown) that could be added after implantation. These living hinges/connectors may be used for attaching the plates 102 together. After the implant 100 is inserted and attached to the endplate 109 of the vertebral body 105, the living hinges or connectors limit the height of the implant 100 and also prevents the spacer element 104 from excessively dislocating.

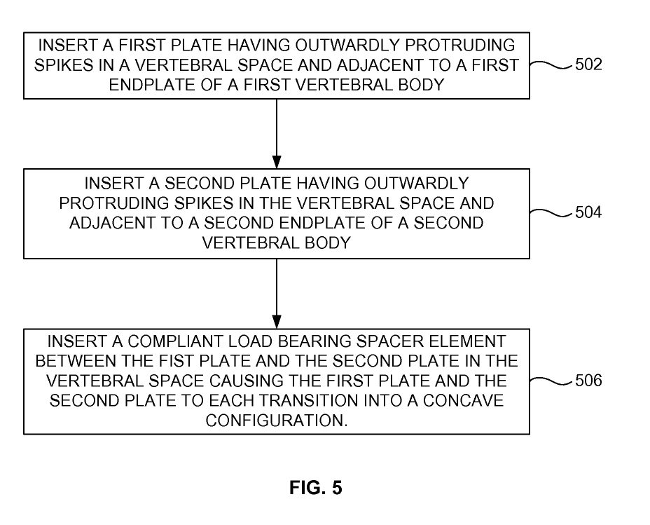

FIG. 5, with reference to FIGS. 1A through 4, is a flow diagram illustrating a method of implanting an artificial vertebral disc according to an embodiment herein. In step 502, a first plate having outwardly protruding spikes is inserted in a vertebral space and adjacent to a first endplate of a first vertebral body. The first plate may be in a convex configuration. In step 504, a second plate having outwardly protruding spikes is inserted in the vertebral space and adjacent to a second endplate of a second vertebral body such that a gap exists between the first plate and the second plate. The second plate is in a convex configuration. In step 506, a compliant load bearing spacer element is inserted in between the fist plate and the second plate in the vertebral space causing the first plate and the second plate to each transition into a concave configuration. In step 508, outwardly protruding spikes may be embedded into the first vertebral body and the second vertebral body when the first plate and the second plate are in the concave configuration.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A multiple-state geometry artificial disc assembly attached to vertebrae, said assembly comprising:
a compliant load bearing spacer element having an upper curved portion and a lower curved portion;
a first plate coupled on said upper curved portion of said compliant load bearing spacer element; and
a second plate coupled on said lower curved portion of said compliant load bearing spacer element;
wherein said first plate and said second plate comprise a flexible material, wherein said first plate and said second plate transitions from a convex configuration to a concave configuration in-situ in a vertebral disc space.

2. The assembly of claim 1, wherein said upper curved portion and said lower curved portion of said compliant load bearing spacer element comprises a plurality of openings.

3. The assembly of claim 1, wherein said compliant load bearing spacer element further comprises a middle cylindrical portion dimensioned and configured to match a gap between said first plate and said second plate.

4. The assembly of claim 1, wherein said first plate and said second plate comprise a plurality of spikes on at least one surface of said first plate and said second plate.

5. The assembly of claim 4, wherein said spikes embed into said vertebrae.

6. The assembly of claim 1, wherein said flexible material comprises any of a polymer, a metal, and a nitinol shaped memory alloy.

7. The assembly of claim 1, wherein said compliant load bearing spacer element comprises any of a flexible polymer material, a polymer, and a hydro-gel.

8. An apparatus to restore a spinal segment mobility comprising, said apparatus comprising:
a first plate comprising flexible material;
a second plate comprising flexible material; and
a compliant load bearing spacer element positioned between said first plate and said second plate, wherein said compliant load bearing spacer element comprises an upper curved portion, a middle cylindrical portion, and a lower curved portion,
wherein each of said first plate and said second plate comprises:
a top surface comprising at least one spike extending outwardly from said top surface;
a bottom surface;
a wall configured around a circumference of said first plate and said second plate such that said wall separates said top surface from said bottom surface; and
at least one gap dispersed along said wall,
wherein said first plate and said second plate transition from a convex configuration to a concave configuration, and
wherein said compliant load bearing spacer element causes said transition of said first plate and said second plate from a convex configuration to a concave configuration to occur in-situ in a vertebral disc space.

9. The apparatus of claim 8, wherein said compliant load bearing spacer element controls at least one of a rigid rotation, a translation, and an active spring plus damping of vertebral bodies.

10. The apparatus of claim 8, wherein said compliant load bearing spacer element comprises a monolithic mass insert-molded around another body of a varied geometry.

11. The apparatus of claim 8, wherein said compliant load bearing spacer element comprises a dual durometer material.

12. The apparatus of claim 11, wherein said dual durometer material controls at least one of a flexion, an extension, a rotation, and a translation of vertebral bodies.

13. The apparatus of claim 8, wherein said compliant load-bearing spacer element comprises a plurality of openings.

14. The apparatus of claim 8, wherein said first plate and said second plate comprise any of a polymer, a metal, and a nitinol shaped memory alloy.

15. A method of implanting an artificial vertebral disc, said method comprising:
inserting a first plate comprising outwardly protruding spikes in a vertebral space and adjacent to a first endplate of a first vertebral body, wherein said first plate is in a convex configuration;
inserting a second plate comprising outwardly protruding spikes in said vertebral space and adjacent to a second endplate of a second vertebral body such that a gap exists between said first plate and said second plate, wherein said second plate is in a convex configuration; and
inserting a compliant load bearing spacer element in between said fist plate and said second plate in said vertebral space causing said first plate and said second plate to each transition into a concave configuration.

16. The method of claim 15, wherein said compliant load bearing spacer element comprises
an upper curved portion comprising a plurality of openings;
a middle cylindrical portion dimensioned and configured to match a configuration of said gap between said first plate and second plate; and
a lower curved portion comprising a plurality of openings.

17. The method of claim 15, wherein said first plate and second plate comprise any of a polymer, a metal, and a nitinol shaped memory alloy.

18. The method of claim 15, further comprising embedding said outwardly protruding spikes into said first vertebral body and said second vertebral body when said first plate and said second plate are in said concave configuration.

19. The method of claim 15, wherein said compliant load bearing spacer element controls at least one of a rigid rotation, a translation, and an active spring plus damping of said first vertebral body and said second vertebral body.

20. The method of claim 15, wherein said compliant load bearing spacer element comprises any of a flexible polymer material, a polymer, and a hydro-gel.
